(19) United States
(12) Patent Application Publication
(10) Pub. No.: US 2023/0149921 A1
(43) Pub. Date: May 18, 2023

(54) Microfluidic System for Diesel Detection

(71) Applicant: Saudi Arabian Oil Company, Dhahran (SA)

(72) Inventors: Maha Nour, Thuwal (SA); Amjad Felemban, Thuwal (SA); Hamad Al Saiari, Al-Khobar (SA)

(21) Appl. No.: 17/529,956

(22) Filed: Nov. 18, 2021

(51) Int. Cl. B01L 3/00; B01L 7/00; G01N 21/78; G01N 33/28

(52) U.S. Cl. CPC B01L 3/502715; B01L 3/5023; B01L 7/52; G01N 21/78; G01N 33/28; B01L 2400/0442; B01L 2300/18; B01L 2300/08; B01L 2300/0627; B01L 2200/147

(57) ABSTRACT

A system includes a microfluidic device having a substrate, a reservoir defined in the substrate a microfluidic channel formed in the substrate, a microheater, and a detector. The reservoir is configured to store a fluid sample to be tested for the presence or absence of a compound. The microfluidic channel extends from the reservoir and includes a first portion fluidically connected to the reservoir, a second portion, and a detection portion fluidically connected between the first and second portions. The microheater is arranged adjacent to the reservoir and is configured to heat the fluid sample to a temperature at which the fluid sample releases a byproduct in response to being heated. The detector is arranged in the detector portion and is configured to indicate a presence or absence of the compound in the byproduct released from the heated sample.

### TECHNICAL FIELD

This disclosure relates to systems and methods for detecting a compound in a sample using a microfluidic device.

### BACKGROUND

Downstream petroleum theft, adulteration, dilutions, and other petroleum related-crimes are often combatted using fuel marking programs. Petroleum products, particularly those containing diesel, might be tampered with at individual retail stations, during the transit link from storage depots, and at the storage depots. Molecular marker additives can attach to diesel and can be detected using complex measuring instruments, such as gas chromatography or X-ray fluorescence (XRF) spectrometers. The measuring instruments are able to detect diesel in low concentrations depending on the marker additive concentration ratio to the fuel. Other counter exploitation technologies such as digitized metering, GPS tracking, aerial surveillance, and other complex monitoring platforms are also used to combat downstream petroleum crimes.

### SUMMARY

This disclosure relates to systems and methods for detecting a compound in a sample using a microfluidic device.

In certain aspects, a system includes a microfluidic device with a substrate, a reservoir defined in the substrate, a microfluidic channel formed in the substrate, a microheater arranged adjacent to the reservoir, and a detector. The reservoir is configured to store a fluid sample configured to be tested for the presence or absence of a compound. The reservoir has a reservoir outlet. The microfluidic channel extends from the reservoir outlet of the reservoir. The microfluidic channel includes a first portion fluidically connected to the outlet of the reservoir, a second portion, and a detection portion arranged between the first portion and the second portion. The detection portion is fluidically connected to the first portion and the second portion. The microheater is configured to heat the fluid sample to a temperature at which the fluid sample releases a byproduct in response to being heated. The detector is arranged in the detection portion and is configured to indicate a presence or absence of the compound in the byproduct released from the heated sample.

In some systems, the microfluidic channel has a channel inlet that is fluidically coupled to the reservoir, and a channel outlet that is fluidically coupled to the detection portion.

In some embodiments, the byproduct released by the heated sample is configured to flow through the microfluidic channel. Some byproducts released by the heated sample are gaseous byproducts. Some gaseous byproducts are configured to flow through the detection portion of the microfluidic channel. Some gaseous byproducts are configured to flow from an inlet of the microfluidic channel to an outlet of the microfluidic channel.

In some systems the microheater is arranged in the reservoir.

Some detectors include a pH detection medium. The pH detection medium may be configured to indicate a change in pH by a visual change, for example a color change. In some detectors, the pH detection medium is a bicarbonate indicator solution.

In some systems, the detection portion of the microfluidic channel includes a U-shaped section. The detector can be arranged adjacent to and/or in the U-shaped section.

Some systems also include a controller operatively coupled to the microheater. The controller includes one or more processors and a computer-readable medium storing instructions executable by the one or more processors to perform operations. The operations includes transmitting an instruction to the microheater to cause the microheater to heat the reservoir to a first temperature; and prompting the microheater to heat the reservoir to a second temperature.

Some systems also include a controller with one or more processors and a computer-readable medium storing instructions executable by the one or more processors to perform operations. The operations include detecting a predetermined level of a compound, based on a pH measurement or color change.

Some systems include a detector with a pH detection medium and a controller with one or more processors and a computer-readable medium storing instructions executable by the one or more processors to perform operations. The operations include prompting an imaging subsystem to analyze a color or color change exhibited by the pH detection medium and detecting a predetermined level of a compound, based on the color or color change.

In some embodiments, the system has a pH sensor arranged in the detection portion of the microfluidic channel and the pH sensor is configured to measure a pH or a change in pH of the detector. Some detectors include a porous medium arranged in the detection portion of the microfluidic channel. A pH detection medium can be contained in the porous medium.

In some systems, the sample includes a combustible compound. The combustible compound may be diesel.

Some systems also include an imaging subsystem configured to image or scan the detector in the detection portion of the microfluidic channel. The imaging subsystem can include a camera.

In certain aspects, a method to detect a diesel concentration in a sample is disclosed. The method includes heating, by a microheater, a sample in a reservoir of a microfluidic device such that the sample releases a byproduct and the byproduct flows through a microfluidic channel connected to the reservoir and detecting, by a change in pH of a detector in a detection portion of the microfluidic channel of a microfluidic device, an amount of the byproduct released from the heated sample.

In some methods, heating, by the microheater, a sample in the reservoir of the microfluidic device such that the sample releases the byproduct and the byproduct flows through a microfluidic channel connected to the reservoir, includes heating, by the microheater, the sample in the reservoir to a first temperature, and heating, by the microheater, the sample in the reservoir to a second temperature. The sample can include a combustible compound. The first temperature can be an auto-ignition temperature of the combustible compound. The second temperature may be a flashpoint temperature of the combustible compound. The combustible compound can be diesel.

In some methods, the first temperature is about 49° C. to about 52° C.

In some embodiments, the second temperature is about 200° C. to about 220° C.

Some byproducts are CO₂. In some methods, an amount of CO₂ released from the sample is proportional to the amount of a combustible compound in the sample.

In some methods, a pH sensor arranged in the detection portion detects the change in pH.

In some embodiments, the detector includes a pH detection medium wherein the pH detection medium visually indicates that a pH change has occurred. The pH detection medium may change color at a predetermined pH. The pH detection medium can be bicarbonate indicator solution.

Some methods also include determining a presence or absence of byproduct released by the heated sample based on the detected change in pH.

Some methods also include determining a presence of a compound in the sample based on the presence or absence of the byproduct.

In some embodiments, the method also includes determining an amount of byproduct released by the heated sample. Some methods include determining an amount of a compound of the sample based on the amount of byproduct.

In some methods, the sample is a portion of a refined petroleum product.

The details of one or more embodiments are set forth in the accompanying drawings and the description below. Other features, objects, and advantages will be apparent from the description and drawings, and from the claims.

### DESCRIPTION OF DRAWINGS

FIG. 1 is a perspective view of a system having microfluidic device to detect the presence of a compound in a sample.

FIG. 2 is a cross sectional side view of a reservoir, microheater, and microfluidic channel of the microfluidic device in use.

FIG. 3 is a graph of temperature pattern of the microheater.

FIG. 4 is a flow chart of a method to detect the presence or absence of a compound in a sample using a microfluidic device.

Like reference symbols in the various drawings indicate like elements.

### DETAILED DESCRIPTION

This disclosure relates to a method and microfluidic system to detect the presence, absence, or amount of a compound in a sample of a refined petroleum product (e.g., a lubrication-oil mixture). The compound can be, for example, diesel, however the techniques describe herein can be implemented to detect other compounds in other samples. The system includes a microfluidic device having a substrate in which a microfluidic channel and reservoir are defined. The reservoir holds a volume, e.g., about 1 microliters (μL) to about 1 milliliters (mL) of a sample taken from the lubrication-oil mixture. A microheater in the microfluidic device heats the reservoir so that the sample releases a gaseous byproduct at an amount proportional to the amount of the compound in the sample. The byproduct rises out of the reservoir and enters the microfluidic channel. The microfluidic channel includes a detection portion 106c at which the presence, absence, and/or, if present, an amount of the byproduct is detected by a detector. The presence of a byproduct indicates that the compound is present in the sample whereas the absence of the byproduct indicates that the compound is not present in the sample. In some cases, the amount of byproduct released can be measured, and the amount of compound in the sample can be calculated based on the amount of byproduct measured.

In the case where the compound to be detected is diesel, the byproduct is carbon dioxide (CO₂) which is released when diesel is heated. The microheater initially heats the sample to a first temperature (e.g., an auto-ignition temperature of diesel) to ignite the diesel; then, reduces the heat applied to sample at a lower second temperature (e.g., the flashpoint temperature of diesel). In the lubrication-oil mixture, diesel has the lowest flashpoint and auto-ignition temperature compared to the common additives and lubricants present in lubrication-oil mixture. The temperatures are selected to ensure that only diesel ignites and continues to combust. The byproduct, CO₂, is generated due to the combustion of only diesel and the amount of CO₂ released is proportional to the amount of diesel in the sample. The CO₂ flows into the detection portion 106c of the microfluidic channel, and a detector arranged in the detection portion 106c indicates a presence, amount, predetermined amount, or absence of CO₂.

Not detecting the presence of diesel (the absence of diesel) in the refined petroleum product can indicate the refined petroleum product from which the sample was taken has been tampered with, without the use of expensive molecular marker additives. Similarly, the detection of diesel in the refined oil sample can confirm that the refined petroleum product has not been tampered with or diluted, without the use of expensive marker additives. Further, some systems confirm predetermined amounts of diesel in the sample and/or measure the amount of diesel in the sample. The methods and systems described herein allow an operator to test the refined petroleum product at the storage site or in the field, without expensive and complicated equipment, for examples a gas chromatography or X-ray fluorescence (XRF) spectrometer. The microfluidic systems have a low power consumption, use a small testing sample volume, and provide results quickly. The system can reduce downstream petroleum crime by identifying quickly in the field, if downstream petroleum crime has occurred. The system can also be used to identify assets or identify materials, for example, a martial fluid that includes a substance mixed with material when the substance has a lower auto ignition temperature than the material.

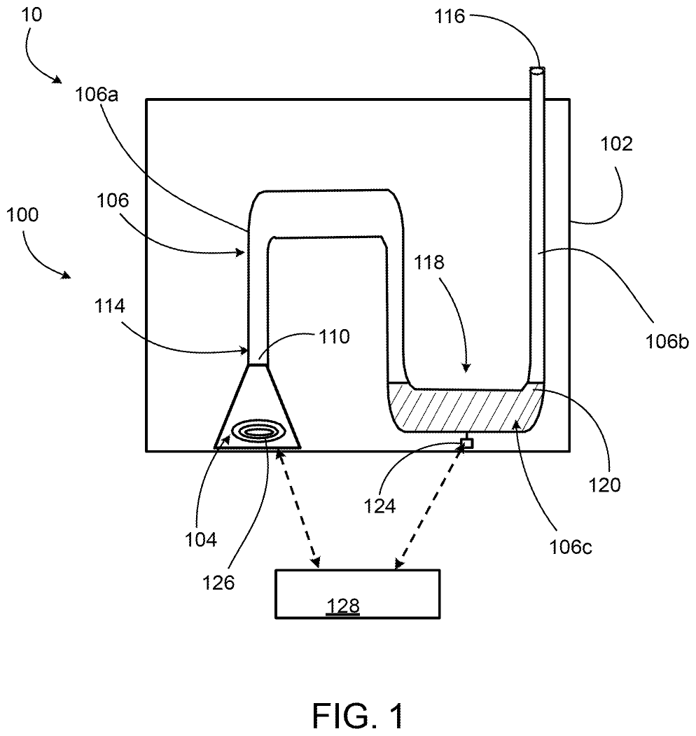

FIG. 1 is a perspective view of a system 10 having microfluidic device 100 to detect the presence of a compound in a sample. The microfluidic device 100 includes a substrate 102 made of glass, plastic, silica, or paper. In some cases, the device 100 is formed entirely on just one substrate. In some cases, the device 100 is formed by bonding two substrates together. The substrate 102 defines multiple recesses, including a reservoir 104 and a microfluidic channel 106. In some systems, the reservoir is part or an extension of the microfluidic channel.

The microfluidic chip can be fabricated by a variety of different methods. For example the microfluidic channel in the microfluidic chip can be formed by wet etching, by Reactive ion etching (ME), or by a laser a glass wafer or glass sheet. The microfluidic channel 106 is formed by laser ablation on a first glass wafer (glass sheet), creating a trench a trench in the first glass wafer. The reservoir is also formed by the same process as the microfluidic channel. In some systems, the reservoir is part or an extension of the microfluidic channel and is formed while etching the microfluidic channel. The reservoir may be defined in the first glass wafer and/or the second glass wafer. In some cases, the reservoir and channel are formed by different processes. In some cases the microfluidic channel is formed in the second wafer or the first wafer and the second wafer. The first glass wafer is encapsulated and anodic bonded with second glass sheet, on which a microheater 105 has been fabricated or fabricated. The microheater 105 on the second glass wafer is aligned with the reservoir 104 defined in the first glass wafer. The microfluidic channel 106 and reservoir 104 are empty from fluids. A detector can be injected or loaded into the microfluidic chip 100 via the outlet. In some cases, the detector is loaded into the microfluidic chip prior to bonding the wafers together.

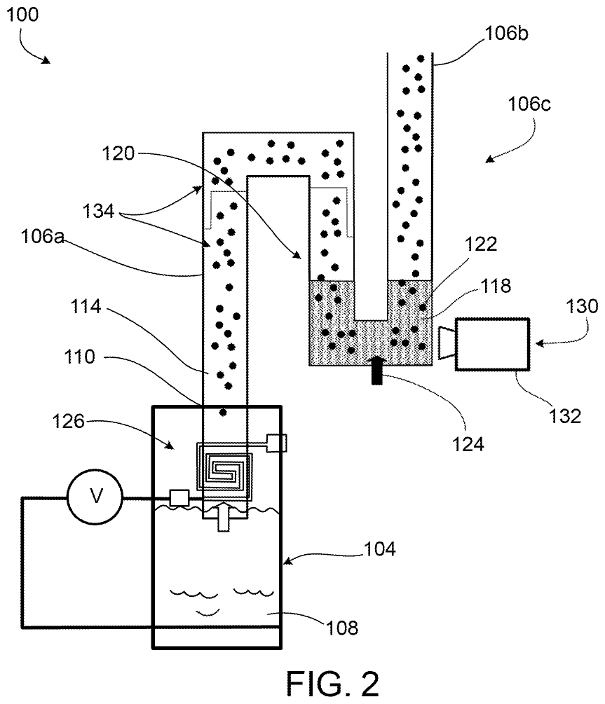

The reservoir 104 stores a fluid sample 108 (FIG. 2), for example, a hydrocarbon mixture or other fluid source. The fluid sample 108 is tested for the presence or absence of a compound, for example diesel. The compound can be a combustible compound (e.g., diesel) having a flashpoint temperature and an auto-ignition temperature. The compound can be any combustion material in a fluid sample (mixture solution) with lower auto-ignition temperature compared to the mixture solution. The reservoir 104 has a reservoir outlet 110 that fluidly connects the microfluidic channel 106 to the reservoir 104. The reservoir outlet 110 is arranged at the top of the reservoir 104 so that a gaseous byproduct rises to enter the microfluidic channel 106, separating the fluid sample from the gaseous byproduct.

The microfluidic channel 106 is an elongated channel that has a channel inlet 114 and a channel outlet 116. The channel outlet can connect to the environment, to exhaust the gaseous byproduct or can connect to a byproduct reservoir (not shown) that holds the byproduct after detection. The elongated microfluidic channel 106 has multiple portions that are each a section of the elongated microfluidic channel 106. The microfluidic channel 106 includes a first portion 106a fluidically connected to the reservoir outlet 110 of the reservoir 104, a second portion 106b, and a detection portion 106c arranged between the first portion 106a and the second portion 106b. The detection portion 106c is fluidically connected to the first portion 106a and the second portion 106b. The channel inlet 114 is fluidically coupled to the first portion 106a of the microfluidic channel 106 and the channel outlet 116 is fluidically coupled to the second portion 106b. When the sample is heated and releases a byproduct, the microfluidic channel 106 flows the gaseous byproduct from the channel inlet 114, to the first portion 106a, through the detection portion 106c, through the second portion 106b, and out the channel outlet 116.

The detection portion 106c of the microfluidic channel 106 includes a U-shaped section 118 in which a detector 120 is arranged. The U-shaped section 118 is oriented so that the detector is at the valley of the “U” when the microfluidic device 100 is upright and positioned correctly for use. The U-shaped section 118 has a first vertical arm and a second vertical arm connected together by a horizontal arm to form a valley in which the detector 120 is disposed. In some detection portions, the detector is arranged in the first vertical arm, the second vertical arm, and/or the horizontal arm. Some detectors extend through the entire U-shaped section. The detector 120 may be adjacent to the U-shaped section such that the detector is aligned with the U-shaped section, outside the channel (detection portion). The detector 120 indicates a presence or absence of the compound based on the byproduct released from the heated sample.

The U-shaped section 118 is oriented so that the detector is at the valley of the “U” when the microfluidic device 100 is upright and positioned correctly for use. In use, the microfluidic device 100 is oriented so that the reservoir 104 is arranged below the reservoir outlet 110. This orientation allows the gaseous byproduct to be separated from the fluid sample when the fluid sample is heated by the microheater. In some cases, the sample is arranged below the reservoir outlet but the reservoir outlet is arranged in a sidewall of the reservoir. In the correct orientation of the microfluidic device 100, the channel 106 extends vertically upward from the reservoir 104. Some microfluidic devices can have different orientations.

The detector 120 includes a pH detection medium 122. The pH detection medium can be bicarbonate indicator solution or a nondispersive infrared sensor. The pH detection medium 122 is sensitive to changes in pH and can produce a visual indication that the pH has increased or decreased. The visual indication can be a color change, transparency change, color intensity change, bubbling, or coagulating. The color change can depend on the type of pH detection medium, for example, a bicarbonate (indicator) solution is red in the neutral status, purple in the base status, and yellow in the acidic status. Other pH detection mediums may be blue as the neutral PH level and a different color in the acidic status. In some cases, the pH sensitive medium does not provide a visual indication that the pH has increased or decreased. In some cases, the detector includes a porous medium (e.g., a sponge) that contains the pH detection medium.

The device 100 also includes a pH sensor 124 arranged in the detection portion 106c of the microfluidic channel 106. The pH sensor 124 measures a pH or a change in pH of the pH detection medium 122. Some detectors include the pH sensor. The change in pH is due to the byproduct from the heated sample flowing through the detection portion 106c. In some cases, other sensors can be arranged in the detection portion 106c, for example, humidity sensors, nitrogen sensors, oxygen sensors, carbon dioxide sensors, methane sensors, sulfur sensors, sulfur dioxide sensors, nitrogen dioxide sensors, nitric oxide sensors, ammonia sensors, volatile organic compound sensors, hydrocarbon sensors, and/or carbon monoxide sensors.

The device 100 has a microheater 105 arranged adjacent to the reservoir 104 such that the microheater 105 controls the temperature of the reservoir 104 and the fluid sample 108 in the reservoir 104. The microheater 105 is fabricated using a Lift-off lithograph process on a glass wafer substrate (the second glass wafer). The microheater 105 is connected to a power source that applies a voltage to a (thin film) platinum spiral. The applied voltage of the power source controls the temperature of the spiral. The maximum temperature of the microheater depends on the spiral length, resistance, and the applied voltage. The microheater 105 has a maximum temperature of about 210° C. Some microheaters have a maximum temperature of about 150° C. to about 300° C. The microheater 105 is arranged in the reservoir 104 after the first glass wafer is bonded to the second glass wafer. The microheater can be arranged above the reservoir, below the reservoir, on the side of the reservoir, in the reservoir, or any combination thereof. Some heaters may be arranged in walls of the reservoir. In some cases, the reservoir is heated by an external heater rather than a microheater of the device. The microheater 105 heats the fluid sample 108 to a temperature at which the fluid sample 108 releases a byproduct in response to being heated. The microheater 105 is a thin-film spiral heater. Some microheaters are flat heaters covering high surface area of the fluid reservoir 104. Some microheaters have a thickness of about 0.5 nm to about 100 nm.

The system 10 has a controller 128 operatively coupled to the microheater 105 and the pH sensor 124. The controller 128 includes one or more processors and a computer-readable medium (for example, non-transitory computer-readable medium) storing instructions executable by the one or more processors to perform operations. The operations can include transmitting an instruction to the microheater to cause the microheater to heat the storage unit to a first temperature and transmitting an instruction to the microheater to cause the microheater to heat the storage unit to a second temperature. The operations can also include detecting a predetermined level of a compound, based on a visual change or measurement observed by an imaging subsystem or sensor, respectively. The color change may be exhibited by the pH detection medium. The controller 128 detects a predetermined level of the compound, based on the measurement or visual change (e.g., color or color change).

FIG. 2 is a cross sectional side view of a reservoir 104, microheater 105, and microfluidic channel 106 of the microfluidic device 100 in use. The microheater 105 has ignited the compound at the auto-ignition temperature and continues to heat the sample 108 at the flashpoint of the compound. The heated sample releases the byproduct 134 which rises to the reservoir outlet 110 and enters the microfluidic channel 106. The byproduct 134 passes though the detector 120 and changes the pH of the pH detection medium 122 of the detector 120. Some pH detection mediums become more basic as the byproduct passes through the pH detection medium, for example, a bicarbonate solution, the pH detection medium becomes more acidic as more carbon dioxide passes through the pH detection medium. Some pH detection mediums become more basic as the byproduct passes through the pH detection medium.

The system 10 has an imaging system 130 aligned with the detection portion 106c. The imaging subsystem includes a camera 132 aligned with the detection portion 106c of the microfluidic channel 106. In some cases, the imaging subsystem is arranged in a microscope. The substrate 102 is substantially transparent to observe a color change. In some cases, the substrate is opaque and includes a transparent window at the area surrounding the detection portion 106c. Some systems do not include an imaging subsystem.

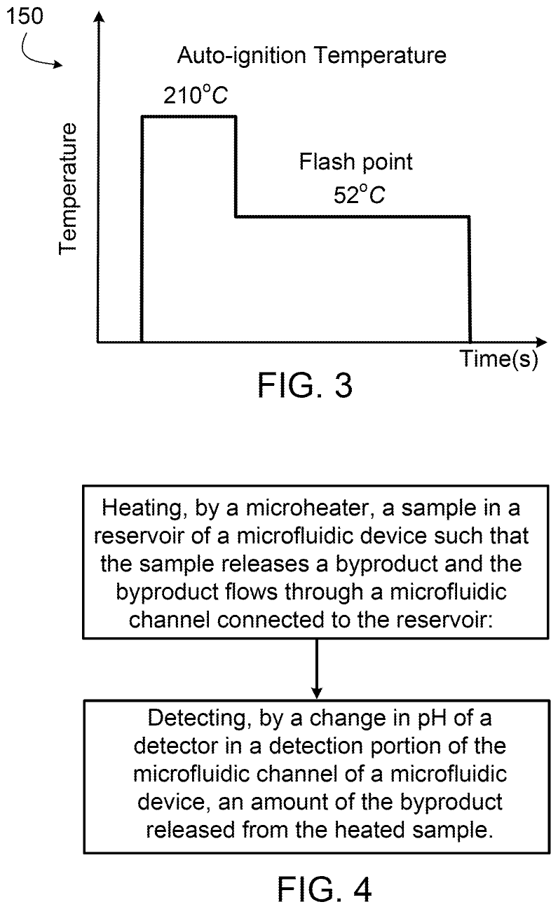

FIG. 3 is a graph 150 of temperature pattern of the microheater 105. The controller 128 prompts the microheater 105 to heat the sample 108 to produce a byproduct 134. In the case that the sample is a lubrication-oi mixture and the compound is diesel, the microheater 105 initially heats the sample 108 to the auto-ignition temperature, about 200° C. to about 220° C., to initially ignite the diesel. The microheater 105 then heats the sample 108 at a second, lower temperature. The second temperature is the flashpoint temperature for diesel, about 49° C. to about 55° C. Heating the sample 108 first by the auto-ignition temperature for a short period of time (e.g., 1 second to 15 minutes, e.g., about 30 seconds to about 5 minutes, e.g., about 1 minutes to about 5 minutes, e.g., about 2 minutes to about 10 minutes, e.g., about 4 minutes to about 6 minutes, e.g., about 2 minutes to about 3 minutes, e.g., 10 seconds, e.g., 20 seconds, e.g., about 30 seconds, e.g., about 45 seconds, e.g., about 60 seconds, e.g., about 90 seconds, e.g., about 2 minutes, e.g., about 2.5 minutes, e.g., about 3 minutes, e.g., about 3.5 minutes, e.g., about 4 minutes, e.g., about 4.5 minutes, e.g., about 5 minutes, e.g., about 5.5 minutes, e.g., about 6 minutes, e.g., about 6.5 minutes, e.g., about 7 minutes, e.g., about 7.5 minutes, e.g., about 8 minutes, e.g., about 8.5 minutes, e.g., about 9 minutes, e.g., about 9.5 minutes, e.g., about 10 minutes, e.g., about 10.5 minutes, e.g., about 11 minutes, or e.g., about 11.5 minutes) begins the combustion of diesel. The temperature is then lowered to the minimum flashpoint temperature so that no other non-diesel components of the lubrication-oil mixture combust and form the byproduct (CO₂). The flashpoint temperature is applied until a majority or all of the diesel in the sample 108 has combusted, about 1 second to about 10 minutes (e.g., about 1 minute to about 5 minutes).

FIG. 4 is a flow chart of a method 200 to detect the presence or absence of a compound in a fluid sample. The method will be described with reference to the system 10 in FIGS. 1 and 2, however the method can be applied to any other applicable system. In addition, the method will be described as testing a hydrocarbon mixture fluid sample for the presence of diesel, however, the system can also be applied to other samples to test for other compounds.

The method 200 includes taking a sample 108 from a fluid source (e.g., a refined petroleum product, lubrication-oil mixture, or hydrocarbon mixture). In some cases, the fluid source is mixed prior to taking a sample so that the fluid source is uniform when the sample is taken. A lubrication-oil mixture that has not been tampered with should have a diesel concentration of about 0.5% to about 100% (e.g., about 5% to about 10%, e.g., about 10% to about 20%, e.g., about 20% to about 30%, e.g., about 30% to about 40%, e.g., about 40% to about 50%, e.g., about 50% to about 60%, e.g., about 60% to about 70%, e.g., about 70% to about 80%, e.g., about 80% to about 90%, and e.g., about 90% to about 100%). If a lubrication-oil mixture has been tampered with the corrupted lubrication-oil will not contain diesel or will contain diesel at a negligible concentration.

The sample 108 is pumped or loaded into the reservoir 104 of the microfluidic channel 106. The sample can be loaded into the reservoir automatically by a pump. The pump can convey a predetermined amount of sample fluid from a fluid source connected to an inlet of the microfluidic chip. In some microchips, the sample can be loaded manually, for example through a syringe injection. The reservoir 104 has an inlet (not shown) through which the fluid sample 108 is inserted. The reservoir 104 has a volume of about 1 μL to about 10 mL (e.g., about 5 ml). The device 100 is oriented so that the reservoir outlet 110 is arranged above the sample 108. The controller 128 then prompts the microheater 105 to heat the sample, containing diesel, to a first temperature, e.g., auto-ignition temperature of diesel. The auto-ignition temperature is used to start the combustion of diesel, producing CO₂, a byproduct 134. The auto-ignition temperature (210° C.) is the lowest required temperature for combustion to start. The fluid source, and therefore the sample, contains many different compounds in addition to diesel, however, the other compounds, for example lubrication oils, each have a higher auto-ignition point than diesel. The most common materials added by smugglers also have a higher auto-ignition temperature than diesel. Applying heat at diesel's auto-ignition temperature will only ignite diesel and the resultant amount of byproduct 134 is directly proportional to the amount of diesel in the sample 108. The microheater 105 continues to heat the sample 108 but reduces the temperature to a second temperature. The second temperature is a flashpoint temperature of diesel, (e.g., about 55° C. to about 65° C.) so that the diesel continues to react and produce. The sample is heated at the second temperature until all the diesel has been reacted, about 1 second to about 10 minutes. If no diesel is present, no carbon dioxide byproduct 134 is released.

The gaseous carbon dioxide byproduct 134 released by the sample upon combustion of the diesel, rises to the ceiling of the reservoir 104 and passes through the filter 112 to enter the microfluidic channel 106 at the channel inlet 114. The carbon dioxide 134 flows through the first portion 106a of the microfluidic channel 106 and enters the detection portion 106c of the microfluidic channel 106. The carbon dioxide flows downward into the U-shaped section 118 of the detection portion 106c where the carbon dioxide interacts with the detector 120. The carbon dioxide naturally flows through the microfluidic chip 100, however, some chips can include or be attached to pumps. The pH detection medium 122 of the detector 120 interacts with the carbon dioxide 134 and the pH of the pH detection medium 122 changes. In some cases, the change in pH of the pH detection medium results in a color change or color intensity change. When testing for the presence of diesel, any change in pH visually alters the pH detection medium 122, resulting in a positive test and indicating that the fluid source has not been adulterated. A positive test occurs when the detector 120 indicates a presence of a byproduct 134, for example, by changing color.

When testing for a predetermined level of diesel, a known amount of carbon dioxide will change the pH to visually alter the pH detection medium. In some cases, a visual change can be or include transparency changes, coagulation, precipitation, a change in volume, or the formation of bubbles.

The method 200 also includes detecting, by a change in the pH of the pH detection medium 122 of the detector 120, an amount, a presence, or an absence of the byproduct 134 released from the heated sample 108. If any carbon dioxide interacts with a pH detection medium 122 (e.g., bicarbonate indicator solution), the pH detection medium 122 changes color (e.g., pink to blue, clear to violet, transparent pink to red) indicating to operator that diesel is present in the sample 108 and in the fluid source. This further indicates that the fluid source has not been tampered with.

In some cases, a pH sensor arranged in the detector or in the detection portion measures a change in pH and the controller detects that the released byproduct is present and that diesel is present in the fluid source. The controller may prompt a display, for example a screen or colored light to activate, indicating to the operator that the fluid sample contains diesel.

In some cases, an imaging subsystem aligned with the detection portion of the microfluidic channel images the detection portion. The controller can analyze the images and determined if a color change has occurred. The controller may prompt a display, for example a screen or colored light to activate, indicating to the operator that the fluid sample contains diesel.

If no diesel is present in the fluid source, and by extension the sample, no carbon dioxide 134 is produced by heating the sample 108. In this case, the pH of the pH detection medium does not change and no color change (or measured change in pH) occurs, resulting in a negative test and indicating to the operator that the diesel is absent from the fluid source and the fluid source has been tampered with. A negative test occurs when the detector does not detect a byproduct 134. In response to a negative diesel test, smuggling countermeasures can be activated, for example digitized metering, GPS tracking, aerial surveillance, and other complex monitoring platforms.

Some smuggling practices dilute the fluid source, rather than removing the diesel completely, resulting in a fluid source with an unintentionally lower concentration of diesel. While the method has been described as testing for the presence or absence of the compound (diesel) in the sample 108, some methods test for a predetermined concentration of diesel in the fluid sample. The method to detect a predetermined concentration of diesel is substantially similar to the method 200, however, the method to detect the predetermined concentration of diesel includes a detector 120 that indicates a positive test to the operator only after the pH has changed a predetermined amount or has reached a threshold.

The method includes testing the sample to determine if diesel is present at a minimum concentration (positive test) or if the diesel is below a minimum concentration (negative test). A positive test (e.g., a color change) occurs when the pH of the pH detection medium has changed by at least a predetermined amount or when the pH of the pH detection medium reaches a threshold pH. A positive test indicates to the operator that the fluid source has not been diluted and the diesel concentration is at an acceptable concentration. A negative test (e.g., no color change) occurs when the pH of the pH detection medium has not changed by at least a predetermined amount or when the pH is below a threshold pH. The negative test indicates to the operator that the fluid source has been diluted and the diesel concentration is below the acceptable minimum concentration.

The pH threshold or predetermined change in pH can be calculated using a known minimum acceptable diesel concentration. The minimum acceptable diesel concentration produces a known amount of CO₂, when fully reacted. The known amount of CO₂ will therefore change the pH of the detector by a predetermined amount. The minimum acceptable diesel concentration can be about 0.5% to about 100%.

As the carbon dioxide flows through the detection portion, the detector reacts with the carbon dioxide to change pH. The detector visually changes only after the pH of the pH detection medium has changed by a predetermined amount or reached a threshold pH. If the detector indicates a positive test, the diesel concentration of the fluid source is at or above the minimum diesel concentration. A positive test is indicated by a color change of the pH detection medium or by a display. If the detector indicates a negative test, the diesel concentration in the fluid source is below the minimum acceptable concentration of diesel. A negative test is indicated by a lack of color change of the pH detection medium or by a display.

In some cases, the amount of diesel in the fluid source can be calculated based on pH measurements taken by the pH sensor in the detection portion of the microfluidic channel. In such a case, the exact change in pH is measured. The change in pH corresponds to an amount of CO₂ released by the sample. The amount of CO₂ that was released from the sample is proportional to the amount of diesel in the sample.

While the diesel has been previously described as completely combusting due to the heat of the microheater, some systems include an oxygen source connected to the reservoir. Oxygen gas can be pumped to the reservoir, prior to or during use, to enhance and support the combustion process. In some cases, the microfluidic chip includes a second inlet fluidly connecting the reservoir to an oxygen source by a channel. The channel may include a valve to automatically or manually control the inflow of oxygen into the reservoir. In some systems, the oxygen is plumps through a second inlet, fluidly connected to the reservoir.

While the detector has been described as providing a visual change to indicate the presence of diesel or a predetermined concentration of diesel, some detectors may be a color sensor or a carbon dioxide detector.

While the system and method have been described to detect a diesel presence, absence, or concentration, other compounds can be tested.

While the system and methods have been described to indicate a concentration of a compound based on a pH change, some systems indicate a concentration of a compound using a byproduct sensor. Some byproduct sensors may be carbon dioxide sensors that identify carbon dioxide levels or change in carbon dioxide levels.

A number of embodiments have been described. Nevertheless, it will be understood that various modifications may be made without departing from the spirit and scope. Accordingly, other embodiments are within the scope of the following claims.

What is claimed is:

1. A system comprising:
a microfluidic device comprising:
a substrate,
a reservoir defined in the substrate, wherein the reservoir is configured to store a fluid sample configured to be tested for the presence or absence of a compound, the reservoir having a reservoir outlet;
a microfluidic channel formed in the substrate, extending from the reservoir outlet of the reservoir, wherein the microfluidic channel comprises:
a first portion fluidically connected to the outlet of the reservoir,
a second portion, and
a detection portion arranged between the first portion and the second portion, wherein the detection portion is fluidically connected to the first portion and the second portion,
a microheater arranged adjacent to the reservoir, the microheater configured to heat the fluid sample to a temperature at which the fluid sample releases a byproduct in response to being heated, and
a detector arranged in the detection portion, wherein the detector is configured to indicate a presence or absence of the compound in the byproduct released from the heated sample.

2. The system of claim 1, wherein the microfluidic channel has a channel inlet that is fluidically coupled to the reservoir, and a channel outlet that is fluidically coupled to the detection portion.

3. The system of claim 1, wherein the byproduct released by the heated sample is configured to flow through the microfluidic channel.

4. The system of claim 3, wherein the byproduct released by the heated sample is a gaseous byproduct.

5. The system of claim 4, wherein the byproduct released by the heated sample is configured to flow through the detection portion of the microfluidic channel.

6. The system of claim 4, wherein the byproduct released by the heated sample is configured to flow from an inlet of the microfluidic channel to an outlet of the microfluidic channel.

7. The system of claim 1, wherein the microheater is arranged in the reservoir.

8. The system of claim 1, wherein the detector comprises a pH detection medium.

9. The system of claim 8, wherein the pH detection medium is configured to indicate a change in pH by a visual change.

10. The system of claim 9, wherein the visual change is a color change.

11. The system of claim 8, wherein the pH detection medium is a bicarbonate indicator solution.

12. The system of claim 1, wherein the detection portion of the microfluidic channel comprises a U-shaped section.

13. The system of claim 12, wherein the detector is arranged adjacent to the U-shaped section.

14. The system of claim 12, wherein the detector is arranged in the U-shaped section.

15. The system of claim 1, wherein the system further comprises a controller operatively coupled to the microheater, the controller comprising:
one or more processors; and
a computer-readable medium storing instructions executable by the one or more processors to perform operations comprising:
transmitting an instruction to the microheater to cause the microheater to heat the reservoir to a first temperature; and
prompting the microheater to heat the reservoir to a second temperature.

16. The system of claim 1, wherein the system further comprises a controller comprising:
one or more processors; and
a computer-readable medium storing instructions executable by the one or more processors to perform operations comprising:
detecting a predetermined level of a compound, based on a pH measurement or color change.

17. The system of claim 1, wherein the detector comprises a pH detection medium, wherein the system further comprises a controller comprising:
one or more processors; and
a computer-readable medium storing instructions executable by the one or more processors to perform operations comprising:
prompting an imaging subsystem to analyze a color or color change exhibited by the pH detection medium; and
detecting a predetermined level of a compound, based on the color or color change.

18. The system of claim 1, further comprising a pH sensor arranged in the detection portion of the microfluidic channel, wherein the pH sensor is configured to measure a pH or a change in pH of the detector.

19. The system of claim 18, wherein the detector comprises a porous medium arranged in the detection portion of the microfluidic channel.

20. The system of claim 19, further comprising a pH detection medium contained in the porous medium.

21. The system of claim 1, the sample comprises a combustible compound.

22. The system of claim 21, wherein the combustible compound is diesel.

23. The system of claim 1, further comprising an imaging subsystem configured to image or scan the detector in the detection portion of the microfluidic channel.

24. The system of claim 23, wherein the imaging subsystem comprises a camera.

25. A method to detect a diesel concentration in a sample, the method comprising:
heating, by a microheater, a sample in a reservoir of a microfluidic device such that the sample releases a byproduct and the byproduct flows through a microfluidic channel connected to the reservoir; and
detecting, by a change in pH of a detector in a detection portion of the microfluidic channel of a microfluidic device, an amount of the byproduct released from the heated sample.

26. The method of claim 25, wherein heating, by the microheater, a sample in the reservoir of the microfluidic device such that the sample releases the byproduct and the byproduct flows through a microfluidic channel connected to the reservoir, comprises:
heating, by the microheater, the sample in the reservoir to a first temperature, and
heating, by the microheater, the sample in the reservoir to a second temperature.

27. The method of claim 26, wherein the sample comprises a combustible compound.

28. The method of claim 27, wherein the first temperature is an auto-ignition temperature of the combustible compound.

29. The method of claim 27, wherein the second temperature is a flashpoint temperature of the combustible compound.

30. The method of claim 27, wherein the combustible compound is diesel.

31. The method of claim 26, wherein the first temperature is about 49° C. to about 52° C.

32. The method of claim 26, wherein the second temperature is about 200° C. to about 220° C.

33. The method of claim 25, wherein the byproduct is CO₂.

34. The method of claim 33, wherein an amount of CO₂ released from the sample is proportional to the amount of a combustible compound in the sample.

35. The method of claim 25, wherein a pH sensor arranged in the detection portion detects the change in pH.

36. The method of claim 25, wherein the detector comprises a pH detection medium wherein the pH detection medium visually indicates that a pH change has occurred.

37. The method of claim 36, wherein the pH detection medium changes color at a predetermined pH.

38. The method of claim 36, wherein the pH detection medium is bicarbonate indicator solution.

39. The method of claim 25, further comprising:
determining a presence or absence of byproduct released by the heated sample based on the detected change in pH.

40. The method of claim 39, further comprising:
determining a presence of a compound in the sample based on the presence or absence of the byproduct.

41. The method of claim 25, further comprising:
determining an amount of byproduct released by the heated sample.

42. The method of claim 41, further comprising:
determining an amount of a compound of the sample based on the amount of byproduct.

43. The method of claim 25, wherein the sample is a portion of a refined petroleum product.
